(19) United States
(12) Patent Application Publication
(10) Pub. No.: US 2002/0193565 A1
(43) Pub. Date: Dec. 19, 2002

(54) Antigen preparation and use

(76) Inventors: Margaret Anne Stanley, Cambridge (GB); Wei Zhang, Cambridge (GB)

Correspondence Address: KLARQUIST SPARKMAN, LLP, One World Trade Center, Suite 1600, 121 S.W. Salmon Street, Portland, OR, 97204, US

(21) Appl. No.: 10/029,430

(22) Filed: Dec. 18, 2001

Related U.S. Application Data
(63) Continuation of application No. 09/647,486, filed on Nov. 9, 2000, now abandoned.
Application No. 09/647,486 is a 371 of international application No. PCT/GB00/00978, filed on Mar. 29, 1999.

(30) Foreign Application Priority Data
Mar. 27, 1998 (GB) 9806666.5

(51) Int. Cl. A61K 39/12; C07K 14/025; C07H 21/04; C07K 2/00; C07K 4/00; C07K 5/00; C07K 7/00; C07K 14/00; C07K 16/00; C07K 17/00; A61K 38/00; C07K 1/00

(52) U.S. Cl. 530/350; 424/186.1; 530/300; 536/23.72

(57) ABSTRACT

Modified virus-like particles (VLPs) can comprise fusion proteins having sequence from a major coat protein of papovavirus, e.g. L1 protein and HPV 16 or 18, in which the N-terminal of the sequence derived from the major coat protein is fused to a further peptide sequence. The VLPs can contain a full sequence of an L1 protein, or an L1 sequence with an N-terminal deletion, or an L1 sequence with an amino acid substitution mutation, and optionally a C-terminal L1 sequence deletion. The peptide sequence fused to the N-terminal can be immunogenic, e.g. from a protein of a pathogen such as a virus. The further peptide sequence can provide a binding domain for affinity purification of the VLP. Modified VLPs can retain the native conformation of the VLP structure while also presenting to the immune system of a subject immunized with the modified VLPs an epitope present on an N-terminal extension of the major coat protein sequence. Corresponding polynucleotides, expression vectors, plasmids, vectors and cells containing such polynucleotides are disclosed.

### FIELD OF THE INVENTION

This invention relates to antigen preparation and use, for example to antigen preparations comprising modified virus-like particles of papovaviruses, e.g. of papillomaviruses. The invention also relates to the production and purification of such antigen preparations, and to corresponding recombinant-nucleic acid constructs e.g. expression cassettes, plasmids, recombinant cells and recombinant viruses applicable to such production. The invention further relates to the use of such antigenic preparations in the production of immune responses, and to vaccines based on such preparations.

### BACKGROUND OF THE INVENTION

Papillomaviruses are members of the papovavirus class of viruses and human papillomaviruses (HPV) in particular are known as agents of disease. For example, infection with HPV type 6 is associated with condyloma acuminatum (genital warts) and HPV type 16 is believed to be a major risk factor for the development of cervical carcinoma.

Several papillomavirus proteins and derivatives of them have been used or proposed for use in vaccines in relation to diseases associated with papillomaviruses.

The DNA sequences of many of the papillomavirus proteins themselves are well-known: see for example the series ‘Human Papillomaviruses’, eds. G Myers et al, published annually at Los Alamos, N. Mex., USA, by Los Alamos National Laboratory (Theoretical Biology and Biophysics, group T10, mailstop K710). See for example references given in issues for 1994 and 1995.

Heterologous expression of papillomavirus proteins is also per-se well known, using conventional cloning technique. References for example can be found in the issues of Human Papillomaviruses (see above) for 1994 and 1995.

Proteins of many papillomavirus types are known, either directly as proteins, or indirectly by viral DNA sequences that encode them, and some have been expressed by recombinant DNA technique in heterologous expression systems.

In particular, it is known to use eukaryotic expression systems, such as the well-known baculovirus/insect-cell expression system, to express papillomavirus capsid proteins in forms that can assemble into multimeric virus-like particles (VLPs). VLPs have been shown to possess immunogenic conformational epitopes not evident in denatured forms of the corresponding viral proteins. Such virus-like particles comprise the major coat protein L1 in the case of papillomaviruses, and can optionally also comprise the minor coat protein L2.

Fusion proteins have also been made on the basis of papillomavirus L1 protein. For example, fusion proteins have been reported comprising HPV16 E7 peptides fused to the C-terminus of L1, the major capsid protein of HPV16, and these have been previously shown to assemble into chimeric virus-like particles (CVLPs). These E7 peptides have been reported as being internally located in the VLPs (Muller et al., Virology 234, 93-111, 1997).

It remains desirable to provide further immunogenic preparations on the basis of VLPs of papovaviruses such as for example papillomaviruses.

### SUMMARY OF DESCRIPTION OF THE INVENTION

The invention rpovides inter alia a fusion protein comprising a sequence from a major coat protein of a papovavirus, in which the N-terminal of the sequence derived from the major coat protein is fused to a further peptide sequence. Also provided by the invention is a virus-like particle comprising such a fusion protein.

Thus, according to an aspect of the present invention, modified major coat proteins of papovaviruses, in which the N-terminal of the major coat protein is fused to a further peptide sequence, can be produced by recombinant DNA technique. In resulting virus-like particles, which can be described as chimeric VLPs, such further peptides can be displayed in such a way that they are immunogenic. Additionally or alternatively, the displayed peptides can also have sequences that provide specific domains for the (affinity) purification of the VLPs. The resulting modified VLPs can also retain conformational epitopes of the corresponding VLPs based on unmodified coat protein(s). Products of the invention can be useful as antigens.

Papovavirus protein involved in such chimeric VLPs can be for example a papillomavirus protein, e.g. human papillomavirus L1 protein from HPV of type 16 or 18. In alternative examples other papillomavirus L1 protein sequences can be used as the basis of the modified proteins, e.g. human papillomavirus L1 of other HPV types such as for example types 1,2, 6 or 11, or 35 or 45, to name several examples that can be useful sources of immunogens related to human papillomavirus infections against which immunogens and vaccines are desired. Major coat proteins of other papovaviruses can be used as the basis of modification if desired, e.g. the major coat protein of SV40 virus.

For example, HPV L1 fusion protein with an N-terminal sequence extension has been expressed in the per-se known baculovirus system in the form of VLPs, i.e. modified VLPs.

This kind of fusion protein comprises, e.g. in an example described below, L1 protein and fused to its N-terminal a sub-sequence from a further protein, for example a papillomavirus early protein such as HPV E1 or E2, E6 or E7. The sub-sequence is preferably long enough to provide at least one epitope of the further protein, e.g. about 15 residues, such as for example residues 45-60 of the E1 aa sequence, alternatively or additionally residues 384-403 of the E1 aa sequence. The E1 aa 45-60 sequence and the 384-403 sequence are believed to be targets of a dominant antibody response obtainable on immunising mice with full-sequence E1 protein.

An example of the fusion protein can be an L1 protein fused at its N-terminal to a short peptide sequence e.g. of about 6-20 aminoacids, e.g. a his-tag or an epitope recognisable by an antibody. VLPs displaying his-tags can be purified after CVLP assembly, e.g. on an appropriate resin such as nickel-NTA-agarose (Qiagen) or Talon-metal affinity resin (Clontech). Epitopes can provide the basis for affinity purification e.g. on an antibody-conjugated protein-G or protein-A derivatised resin.

(The C-terminal of) the chosen peptide sequence can be fused on to the N-terminal of the L1 sequence. This is achieved by construction of a corresponding encoding polynucleotide and its incorporation into an expression vector and sub-sequence expression by per-se wellknown rDNA methods, e.g. in the baculovirus system as in the example given below.

An example of a VLP preparation based on such a fusion protein has been shown to have the following properties:

it is recognised by an antibody from the immune response against the full-sequence protein of which a sub-sequence was fused to the N-terminal of the L1 protein;

it is recognised by an anti-(L1 conformational epitope) antibody made in known manner against unmodified VLPs of the corresponding HPV type.

Accordingly the invention also provides modified VLPs that (a) retain the native conformation of the structure of the corresponding VLPs based on major coat protein of corresponding unmodified sequence while also (b) presenting to the immune system of a subject immunised with the modified VLPs an epitope present on the N-terminal extension of the major coat protein sequence.

If desired the modified major coat protein can have further features and modifications for example as follows:

The N-terminal extension of the major coat protein sequence can comprise a heterologous protein sub-sequence eg it can comprise the sequence of an epitope that is recognisable by a B-cell, e.g. either a neutralising or a non-neutralising epitope, or an epitope that is recognisable by a T-cell, or more than one epitope of either kind or more than one kind.

‘Heterologous’ in this context means having a sequence other than that which is native to the unmodified N-terminal of the wild-type papovavirus major coat protein most nearly corresponding to the structural protein of the modified (chimeric) VLP preparation according to the example of the invention that is under consideration.

The N-terminal extension can be for example of at least about 10 aminoacids in length and up to about 15 aminoacids in length; it can also be somewhat longer, e.g. up to 30, 40, 50 or sometimes 60 aminoacids in length, or as desired, with the proviso that the extension sequence should not be so long that it disrupts the formation of the VLP structure. This can be judged if desired or if need be by per-se known methods, e.g. by comparative electron-microscopic examination of a preparation of the modified protein made under conditions that correspond to VLP-forming conditions in the case of a coat protein that is similar except that it has a shorter or zero-length N-terminal aminoacid sequence extension.

The normal sequence at the N-terminal of the major coat protein L1 to which a heterologous epitope is added as described herein can also if desired be truncated. Such truncation can be chosen so as not to disrupt the ability of the resulting mutant L1 protein with N-terminal sequence extension to form VLPs. For example, deletion of up to about 10 aminoacids of the N-terminal L1 can result in enhanced formation of VLPs in baculovirus-infected insect cells. The ability of mutant L1 proteins with N-terminal sequence extension to form VLPs can be tested when desired e.g. by the electron-microscopic technique described by way of example below.

Without limitation, the N-terminal extension aminoacid sequence can be chosen e.g. from among the following:

a sub-sequence of HIV envelope glycoprotein gp120, e.g. a sub-sequence from the region aa384 to aa467 of gp120, (as identified by PNAS 85: (1988) 7957-7961, J Virol 64: 2452-2455 and/or referenes cited therein), which sequence has been reported as giving rise to both humoral (antibody) and T-cell proliferative response to HIV protein gp120,

a sub-sequence of HIV envelope glycoprotein gp120, from the region aa428 to aa443 of gp120, (env T1 as identified in Nature 334: 706-708 as a T-cell epitope);

other T-cell- and B-cell-recognised epitopes, such as a poliovirus T-cell epitope as described in J Virol 65 (1991) 711-718 (from positions 103-115 of poliovirus VP1 capsid protein); or a B-cell neutralising epitope from human poliovirus VP1 protein positions aa93-103, as described in Science 233 1986 472-475; or a rhinovirus neutralising epitope from positions aa 153-164 of VP2 protein of HRV2 as described in J Gen Virol 68 1987 315-323 and 2687-2691; or an epitope from herpes simplex virus/parvovirus (hybrid neutralising epitope from gD envelope protein), as described in J Virol 49 1984 102-108 or Virology 198 1994 477-480; or a HIV 38-aa neutralising domain sequence from within the V3 loop of gp120, as described in PNAS 86 1989 6768-6772;

a sub-sequence that facilitates affinity-binding, e.g. a his-tag (six his residues), or any antibody-recognised epitope.

It can be seen that the invention also provides a method of inducing an immune response to a T-cell-recognised epitope or a B-cell-recognised epitope, which comprises immunising a subject to be treated with a preparation of modified papovavirus VLPs in which the major coat protein component has an aminoacid sequence wherein the T-cell epitope or B-cell epitope sequence against which the immune response is to be generated is present in a N-terminal sequence extension of said major coat protein component.

VLP preparations according to the invention, including pharmaceutical formulations e.g. in sterile injectable dosage forms comprising the VLPs along with e.g. per-se known excipients, are expected to provide useful prophylactic and therapeutic immune responses related to epitopes present on the N-terminal aminoacid sequence extensions described above.

The invention is further illustrated by the following description of non-limitative examples and by the accompanying drawings. Indicative contents of the drawings, described in further detail below, are:

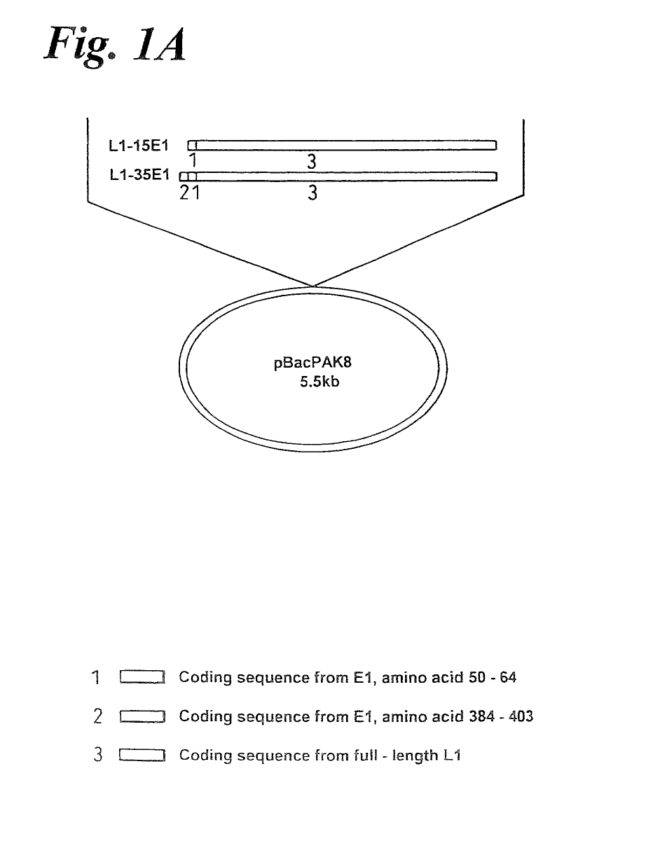

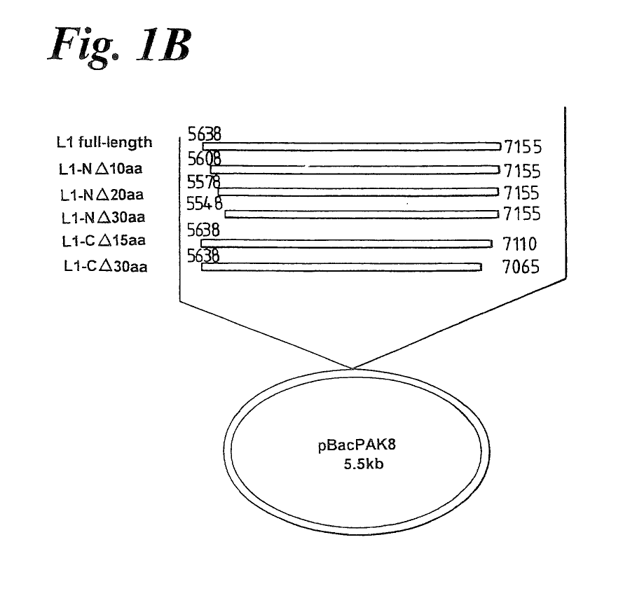

FIG. 1A is a plasmid diagram showing constructs for expressing L1 protein of HPV with fused N-terminal peptides, suitable for making protein preparations according to embodiments of the invention.

FIG. 1B is a plasmid diagram showing constructs for expressing L1 protein of HPV full-length and with N-terminal or C-terminal deletions.

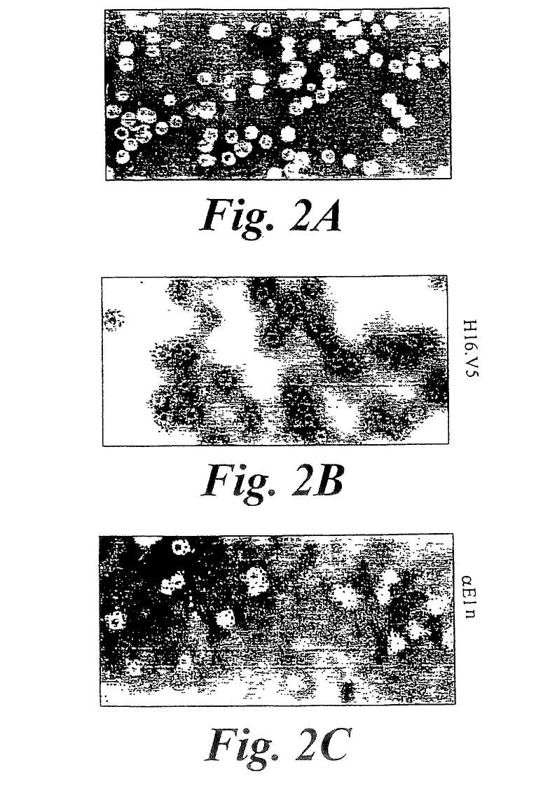

FIGS. 2A, 2B and 2C show electron micrographs of particles formed from one of the protein fusions corresponding to FIG. 1A.

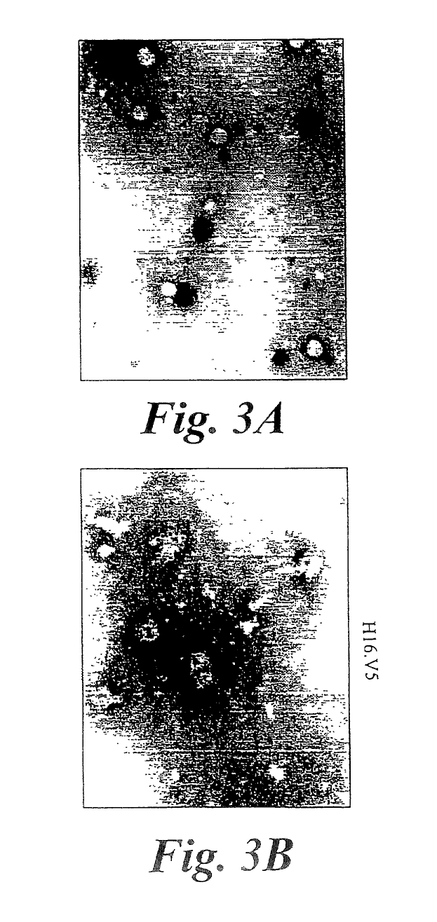

FIGS. 3A and 3B show electron micrographs of particles formed from another of the protein fusions corresponding to FIG. 1A.

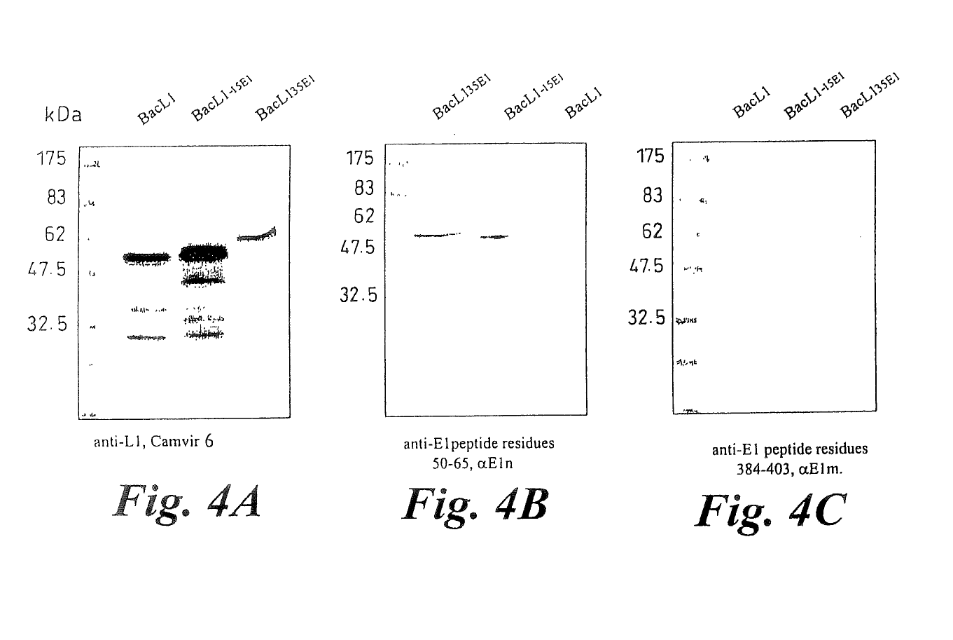

FIGS. 4A, 4B and 4C show Western blots diagnostic of the presence of fusion peptides in protein fusions expressed from the constructs of FIG. 1A.

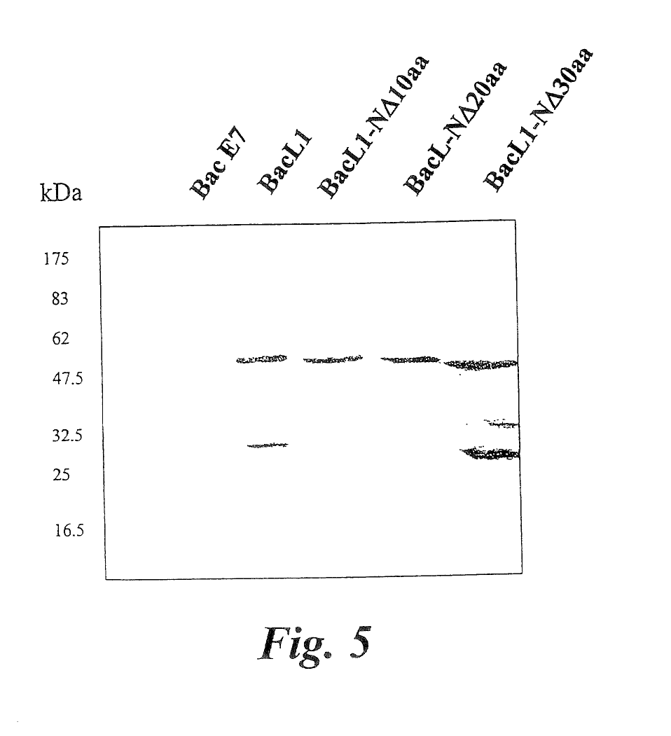

FIG. 5 shows a Western blot indicative of expression of N-terminal truncated proteins corresponding to FIG. 1B.

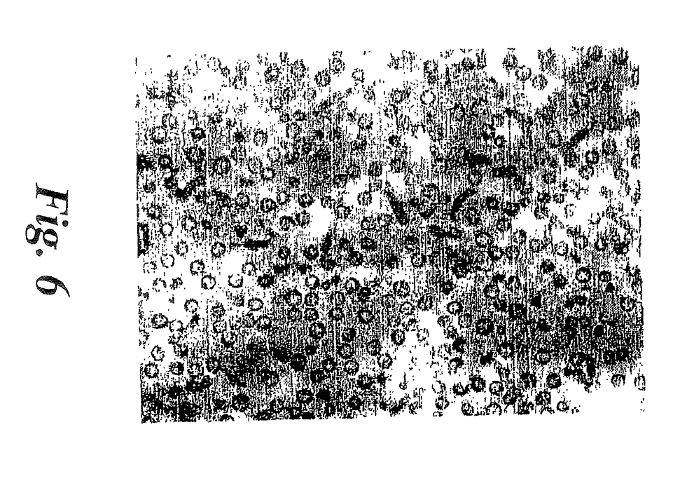

FIG. 6 shows an electron micrograph of VLPs formed from one of the N-terminal truncated proteins corresponding to FIG. 1B.

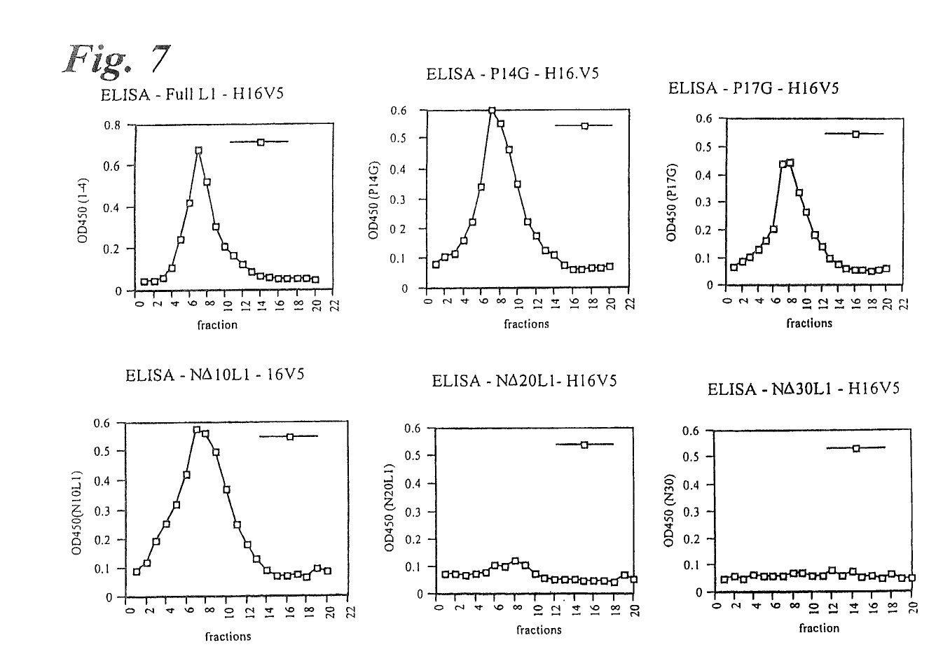

FIG. 7 shows ELISA testing of fractions from expression of N-terminal truncated L1 proteins corresponding to FIG. 1B.

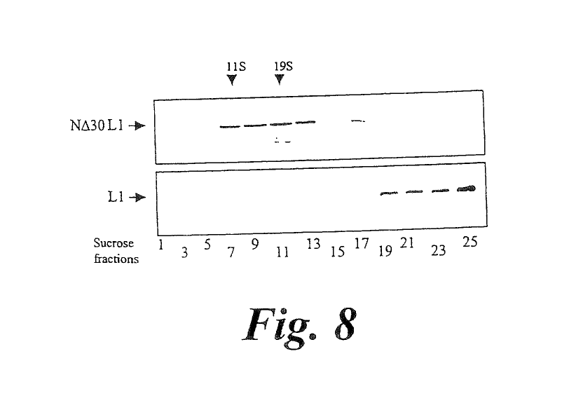

FIG. 8 shows results of density gradient centrifugation of particles formed by one of the N-terminal truncated L1 proteins corresponding to FIG. 1B.

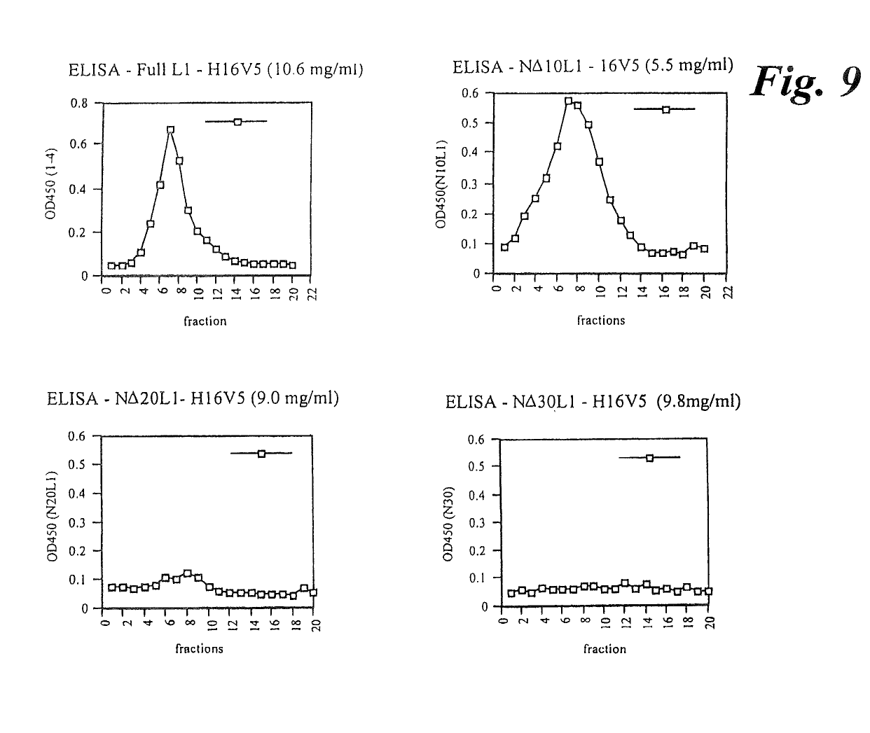

FIG. 9 shows ELISA testing of fractions from expression of aminoacid substitution mutants of L1 protein.

### EXAMPLE

The following description shows generation and characterisation of chimeric particles of human papillomavirus type 16.

### INTRODUCTION

Fusion proteins comprising HPV16 E7 peptides fused to the C-terminus of L1 protein were previously shown to assemble into chimeric virus-like particles. The present example describes fusion of epitopes recognised by respective monoclonal antibodies at the N-terminus of L1 protein and expression of the corresponding hybrid protein in a baculovirus system. Fusion proteins as indicated below were able to self-assemble into chimeric VLPs and fused epitope was found to be presented on the surface of such VLPs, without interfering with the reactivity of the conformation-dependent neutralising epitopes. Thus the N-terminus of the L1 protein is shown to be suitable for epitope fusion and presentation. Chimeric VLPs generated in this and analogous ways can be used for immunisation against dual or multiple epitopes.

### METHODS

### Construction of Baculovirus Transfer Recombinants

Three recombinant plasmids were generated for expression of (A) full-length L1 protein (as a control), and L1 fusion proteins in which respectively (B) the amino (N)-terminus of full-length L1 was fused to aminoacid residues 50-65 from E1 protein of HPV16, and (C) the amino (N)-terminus of full-length L1 was fused to amino acid residues 50-65 from E1 protein of HPV16 and additionally, aminoacid residues 384-403 from E1 protein of HPV16 were fused at the N-terminus corresponding to sequence (B).

For generation of recombinant (A), the sequence of HPV16 L1 ORF was amplified by polymerase chain reaction (PCR) from a total DNA extract of W12, an HPV16 episome-containing cell line (Stanley M, Brown H M, Appleby M, and Minson A C (1989) “Properties of a non-tumorigenic human keratinocyte cell line”, Int J Cancer 43, 672-676) using a forward primer in which a Bgl II restriction sequence was included (underlined) 5′-GCT GCA AGA TCT ATG TCT CTT TGG CTG CCT AG-3′.

For generation of recombinant (B), a DNA nucleotide sequence encoding the E1 50-65 sequence plus a Bgl II restriction sequence (underlined) was introduced just in front of the L1 coding sequence as a forward primer, as follows:

5′-GCT GCA AGA TCT ATG GTA GAT TTT ATA GTA MT GAT AAT GAT TAT TTA ACA CAG GCA GAA TCT CTT TGG CTG CCT AGT GAG-3′.

For generation of recombinant (C), a nucleotide sequence encoding E1 amino acids 384-403 was introduced just in front of E1 50-65 coding sequence of the last-mentioned construct, using a forward primer with a flanking Bgl II restrictional sequence (underlined): 5′-GCT GCA AGA TCT ATG TAC GAT AAT GAC ATA GTA GAC GAT AGT GAA ATT GCA TAT AAA TAT GCA CM TTG GCA GAC GTA GAT TTT ATA GTA AAT GAT-3′.

These forward primers were paired with the same reverse primer in which a Not I restriction site was included (underline). Reverse: 5′-GAT CTA GCG GCC GC TTA CAG CTT ACG CTT CTT GCG TTT-3′.

Following 30 cycles of amplification, the DNA products (e.g. of about 1.7 kb in size) were gel purified, GENECLEAN (TM) excised, digested with restriction enzymes of Bgl II and Not I and sub-cloned into baculovirus transfer vector pBacAK8 (Clontech) which had been pre-digested with BamHI and NotI restriction enzymes. The recombinant plasmids for cases (A), (B) and (C) were examined by sequencing in per-se known manner (Pharmacia (TM) kit).

### Generation of Recombinant Baculoviruses

Insect cells of Spodoptera frugiperda (sf21) were grown in 30 mm dishes until 80% confluent at 27° C. with TNMFH medium (Sigma) supplemented with 10% foetal calf serum. 0.5 micro-g DNA of each respective transfer recombinant together with 5 micro-I of Bsu36 I digested pBacPAK6 viral genomic DNA (Clontech) were co-transfected into the insect cells by following the instruction of the supplier. For the control cells of the transfection, all the agents were applied but viral genomic DNA. Recombinant viruses were plaque purified by per-se known technique and further expanded by multiple cycles of cell infection. To examine in each case whether HPV16 L1 protein or respective L1 fusion protein was expressed, sf21 cells were infected with each of the baculovirus recombinants e.g. at MOI about 5-10. After 72 hrs of infection, cells were harvested, lysed, analysed by SDS-PAGE and immunoblotting with one of the following monoclonal antibodies (mAbs), i.e. anti-L1 mAb Camvir 1 (McLean C S, Churcher M J, Smith G L, Higgins G, Stanley M, and Minson A C (1990) in “Production and characterisation of a monoclonal antibody to human papillomavirus type 16 using recombinant vaccinia virus” J Clin. Pathol. 43, 488-492), or an anti-E1 50-65 amino acid mAb designated alpha-E1n or an anti-E1 384-403 amino acid mAb alpha-E1m. Each of the latter antibodies was expressed by a hybridoma produced by immunising mice with full-length E1 protein of HPV expressed in a standard E coli host expression system, and selecting hybridomas with the ability to produce antibody of the desired specificity using the 50-65 or the 384-403 peptide of E1 protein in per-se known manner.

### Purification of Virus-Like Particles

To purify the full-length L1 VLPs (case A) and respective chimeric VLPs formed with fusion L1 protein (cases B and C) the following procedure was used.

The sf21 cells were monolayer cultured in 175 sq cm tissue culture flasks to 80% confluent, then the culture media were removed from the flasks and the cells were washed once with PBS. Cells for each of cases (A), (B) and (C) were inoculated with respective baculovirus recombinants at a MOI of about 5-10 for 1 hour at room temperature about 27 deg.C. After adding fresh medium into the flask, the cells were postinfected for 3 days at about 27° C. The cells were harvested by centrifugation and each of the cell pellets (containing about 1×10ˆ 8 cells) was resuspended into 15 ml of PBS and homogenised with 50-100 strokes with a Dounce homogenizer (BDH). Nuclei were separated by centrifugation at 2000×g for 20 min and the pellet was resuspended in 4 ml of PBS and mildly sonicated for 30 seconds at mark 18 (Branson Sonifier (TM)) on ice. The nuclei lysate was layered onto a 0.75 ml of 40% (w/v) sucrose-PBS cushion and centrifuged at 34,000 rpm for 2 hours in a Beckman SW55Ti rotor. The pellet was resuspended in 2 ml of PBS followed by a 10 second mild sonication on ice prior to adding 3 ml of high density CsCl/PBS (d=1.5 g/ml). The suspension was centrifuged at 45,000 rpm for 16 hours in a SW55 Ti rotor at 18 deg C. After centrifugation, double-bands of the particles, either VLP's or chimeric VLPs corresponding to fusion proteins (B) or (C), in the middle of the centrifuge tube, were collected by puncturing the tube with a needle. The densities of the collected bands were examined with refractometer (Bellingham & Stanley Ltd. England), and were of e.g. density about 1.30 (top band) to about 1.325 (bottom band). The collected samples were diluted with PBS and pelleted at 34,000 rpm for 2 hours with a SW55Ti rotor. The pellets were dissolved in PBS and stored at 4 deg C.

### Electron Microscopy

To examine the morphological properties of the chimeric VLPs, the particles with a protein concentration of 100-500 micro-g/ml, were spotted onto a glow-discharged carbon coated grid and negative stained with 2% phosphotungstic acid solution (pH 6.8) and viewed with a Phillips CM 100 transmission electron microscope. To further determine the E1 peptides and HPV16 conformational epitopes on the chimeric VLPs, immuno-gold labelling was applied as follows. The grids with the particles were stained with an antibody, either alpha-E1n, or alpha-E1m, or H16.V5 (an HPV16 neutralising mAb specifically recognising the conformational epitope of the virion, as described by N C Christensen, J Dillner, C Eklund, J J Carter, C A Reed, N M Cladel, and D A Galloway (1995): ‘Surface conformational and linear epitopes on HPV16 and HPV18 virus-like particles as defined by monoclonal antibodies’: Virology 223 (1995) 174-184). The antibodies were all diluted 1: 50 in 5% milk/PBST blocking buffer, and incubated at room temperature for 1 hour. The grids were then washed with 1% milk/PBST 3 times at 5 mm for each interval. The grids were then stained with 10 nm gold conjugated anti-mouse IgG (Sigma) at room temperature for 1 hour, then subjected to 3 washes with 1% milk/PBST and 3 washes with distilled water. The grids were then stained with 2% (w/v) phosphotungstic acid pH 6.8) and viewed with a Philips C M 100 (TM) transmission electron microscope.

### Immunisation of Mice

Six-week-old BALB/c mice can be used for immunisation e.g. to test for immunogenicity. The preimmune serum can be sampled prior to immunising the mice e.g. with about 5 micro-g purified VLPs or respective chimeric VLPs in phosphate-buffered saline PBS with or without adjuvant per injection subcutaneously at 3×2 weeks intervals. Serum samples can be collected 3-4 days after each booster immunisation, heat-inactivated e.g. at 56 deg C. for 30 mm, then tested by ELISA.

Properties of constructs useful in carrying out embodiments of the invention are indicated by the accompanying drawings. Referring to the drawings:

FIG. 1A shows a plasmid diagram showing constructs based on commercially available plasmid pBacPAK8 (5.5 kb) with insertion of coding sequences for expressing L1 protein of HPV with fused N-terminal peptides as follows, either 15 aminoacids from HPV E1 protein residues 50-64 (‘L1-15E1′, case B in Example 1), or 20 aminoacids from HPV E1 protein residues 384-403 and 15 aminoacids from HPV E1 protein esidues 50-64 (‘L1-35E1′, case C in Example 1). These fusion proteins are suitable examples for making chimeric VLP preparations according to embodiments of the invention.

FIG. 1B shows plasmid constructs for expressing L1 protein of HPV full-length (starting with base number 5638 and ending with base number 7155 in HPV genome sequence) and with N-terminal deletions of respectively 10, 20 and 30 aminoacid residues (starting with base number 5608, 5578 or 5548 in HPV genome sequence), or C-terminal deletions of respectively 15 and 30 aminoacid residues (ending at base number 7110 or 7065 in HPV genome sequence).

FIGS. 2A, 2B and 2C show electron micrographs of particles formed from protein fusion L1-15E1. FIG. 2A shows negatively-stained particles, FIG. 2B shows particles immuno-stained with mAb H16.V5, specific for HPV16 conformational-dependent epitope, and FIG. 2C shows particles immuno-stained with antibody specific for the aa50-65 epitope of HPV E1 protein. FIG. 2 indicates the formation, from protein L1-15E1, of chimeric VLPs with accessible epitope corresponding to the N-terminal fusion peptide derived from HPV E1 protein.

FIGS. 3A and 3B show electron micrographs of particles formed from protein fusion L1-35E1. FIG. 3A shows negatively-stained particles, and FIG. 3B shows particles immuno-stained with mAb H16-V5 specific for HPV16 conformational-dependent epitope, indicating chimeric VLP formation.

FIGS. 4A, 4B and 4C show Western blots of L1 protein (as control) and of L1-15E1 and L1-35E1 fusion proteins, using ant-L1 antibody (4A), antibody specific for E1 aa50-64 epitope (4B), and antibody specific for E1 384-403 epitope (4C), indicating presence of appropriate respective fusion peptides in protein fusions expressed from the constructs of FIG. 1A

FIG. 5 shows a Western blot indicative of expression of the N-terminal truncated proteins corresponding to FIG. 1B, showing that each protein was expressed and that the molecular weights declined as expected with the deletions.

FIG. 6 shows an electron micrograph of VLPs formed from the N-terminal 10-aa truncated L1 protein corresponding to FIG. 1B, indicating that this deletion did not affect VLP assembly. The yield of VLPs here was increased compared with yield from wild-type L1. VLPs were not seen in corresponding tests with the 21-aa and 30-aa N-terminal deletions.

FIG. 7 shows ELISA testing of fractions from expression of L1 protein (control) and each of the N-terminal truncated L1 proteins corresponding to FIG. 1B. The ELISA used a mAb recognising the conformational epitope in VLPs of HPV16 L1. The conformational epitope was detected in preparations from wild-type L1 and the 10-aa N-terminal deletion only. As the amount of protein expressed was approximately the same in each case, loss of detection of comformational epitope was not due to low protein level.

FIG. 8 shows results of density gradient centrifugation of particles which it was concluded were pentameric structures formed by the 30-aa N-terminal truncated L1 protein corresponding to FIG. 1B, with full-length L1 as control. Total lysates from the expression system were separated on 5-50% sucrose gradients and Western blotting done with anti-L1 antibody. Markers at 11s (catalase) and 19s (beta-galactosidase) indicate pentameric capsomer formation from the 30-aa N-terminal truncated L1 protein (fractions 5-12), whereas fully assembled VLPs are found for full-length L1 protein in fractions 19-25.

FIG. 9 shows ELISA testing of fractions from expression of aminoacid substitution mutants of L1 protein. Mutagenesis of pro(14)-→gly and pro(17)-→gly in the full-length L1 sequence resulted in retention of the ability to form VLPs as assessed by reactivity with antibody to the conformational epitope.

It will be seen that accordingly, the invention in several examples provides chimeric VLPs based on protein fusions comprising N-terminal peptides fused to HPV L1 protein of full sequence, or with N-terminal deletion, e.g. of preferably up to about 10 amino-acids, or with aa substitution mutations, especially those that leave the mutant fusion protein still able to form VLPs Optionally there can be C-terminal deletions of the L1 sequence.

N-terminal fusion proteins contained in chimeric VLPs as described herein can retain the native conformation of the corresponding native VLP structure while also presenting to the immune system of a treated immunised subject whatever epitope is present on the N-terminal extension.

The invention is susceptible of a variety of modifications and variations as will be apparent to the person skilled in the art, and the present disclosure extends to combinations and subcombinations of the features mentioned herein, including the appended claims, and in the cited publications which are hereby incorporated by reference in their entirety.

1. A fusion protein comprising a sequence from a major coat protein of a papovavirus, in which the N-terminal of the sequence derived from the major coat protein is fused to a further peptide sequence.

2: A virus-like particle comprising a fusion protein according to claim 1.

3: A virus-like particle according to claim 2, comprising a sequence from a major coat protein L1 of a papillomavirus, e.g. of HPV type 16 or 18.

4: A virus-like particle according to claim 3, comprising either (i) a full sequence of a human papillomavirus L1 protein, or (ii) a sequence from a human papillomavirus L1 protein having an N-terminal deletion of up to 10 amino-acids, or (iii) a sequence from a human papillomavirus L1 protein with an aminoacid substitution mutation, optionally in each case with a C-terminal deletion of the L1 sequence.

5: A virus-like particle according to claim 3, wherein said further peptide sequence is an immunogenic sequence, e.g. a sequence derived from a protein of a pathogen such as a virus.

6: A virus-like particle according to claim 3, wherein said further peptide sequence provides a binding domain for the (affinity) purification of the virus-like particle.

7: A fusion protein according to claim 1 or a virus-like particle according to claim 3, wherein fused to the N-terminal of a sequence from a papillomavirus L1 protein is a sub-sequence from a further papillomavirus protein, e.g. an early protein such as HPV E1 or E2, E6 or E7.

8: A fusion protein according to claim 1 or a virus-like particle according to claim 2 wherein said peptide sequence is long enough to provide at least one epitope of the further protein, e.g. about 15 residues, for example residues 45-60 of a HPV16 E1 aa sequence, alternatively or additionally residues 384-403 of a HPV 16 aa sequence.

9: A fusion protein according to claim 1 or a virus-like particle according to claim 2 wherein fused at the N-terminal of the major coat protein is a short peptide sequence e.g. of about 6-20 aminoacids, e.g. a his-tag or an epitope recognisable by an antibody.

10: A method of purifying a virus-like particle according to claim 9, by affinity purification on a solid phase with complementary affinity, e.g. nickel-NTA-agarose where the peptide comprises a his-tag.

11: Modified virus-like particles of papovaviruses that (a) retain the native conformation of the structure of the corresponding VLPs based on major coat protein of corresponding unmodified sequence while also (b) presenting to the immune system of a subject immunised with the modified VLPs an epitope present on an N-terminal extension of the major coat protein sequence.

12: Polynucleotides corresponding encoding a fusion protein according to claim 1, and expression vectors, plasmids, vectors and cells containing such polynucleotides.

13: A method of producing fusion proteins according to claim 1 and virus-like particles according to claim 2 which comprises expressing a corresponding encoding polynucleotide according to claim 12 in a host cell expression system, e.g. a eukaryotic expression system such as a baculovirus expression system.
